(19) United States
(12) Patent Application Publication
(10) Pub. No.: US 2014/0378792 A1
(43) Pub. Date: Dec. 25, 2014

(54) ANTI-FOULING SLEEVE FOR INDWELLING CATHETERS

(71) Applicants: William S. Krimsky, Bel Air, MD (US); Murat Yasar, Columbia, MD (US); Gregory J. Hiemenz, Silver Spring, MD (US); Amit Navin Shah, Bethesda, MD (US)

(72) Inventors: William S. Krimsky, Bel Air, MD (US); Murat Yasar, Columbia, MD (US); Gregory J. Hiemenz, Silver Spring, MD (US); Amit Navin Shah, Bethesda, MD (US)

(21) Appl. No.: 14/313,921

(22) Filed: Jun. 24, 2014

Related U.S. Application Data
(60) Provisional application No. 61/838,497, filed on Jun. 24, 2013.

(51) Int. Cl. A61M 16/04; A61B 5/01; A61B 5/03; A61B 5/145; A61B 5/087; A61B 5/1459; A61B 1/07; A61B 1/00; A61L 2/10; A61B 1/06; A61B 5/1473; A61B 5/00; A61B 5/083

(52) U.S. Cl. CPC A61M 16/0427; A61B 5/6852; A61B 5/01; A61B 5/036; A61B 5/14539; A61B 5/14542; A61B 5/087; A61B 5/0833; A61B 5/0836; A61B 1/07; A61B 1/00117; A61L 2/10; A61B 1/0684; A61B 5/1473; A61B 5/1459; A61M 16/0488; A61M 16/0463
USPC 600/310; 128/200.26; 600/300; 600/549; 600/561; 600/361; 600/364; 600/538; 600/532; 600/531; 600/476; 600/249; 600/345

(57) ABSTRACT

An anti-fouling sleeve for an endotracheal tube, a method of placement, and the tools for placement. The anti-fouling sleeve occupies the entire length of endotracheal tube, and can be installed permanently or made removable and disposable. The sleeve may be instrumented with sensors and/or a UV light source to reduce and potentially eliminate biofilm formation. Once placed inside the endotracheal tube the sleeve expands to conform to the inner diameter of the tube. After use, any accumulated biofilm on the inner portion of the sleeve is removed leaving the inner portion of the endotracheal tube essentially sterile.

### CROSS-REFERENCE TO RELATED APPLICATION(S)

The present application derives priority from U.S. provisional application Ser. No. 61/838,497 filed 24 Jun. 2013.

### BACKGROUND OF THE INVENTION

1. Field of the Invention

The present invention relates generally to endotracheal intubation, and more specifically, to a sleeve placed inside an endotracheal tube (ETT) and/or other indwelling catheters (and the tools and methods to do so) which facilitates instrumenting the endotracheal tube with sensors, a UV light source, etc., without risk of potentially damaging biofilm formation.

2. Description of the Background

Endotracheal tubes are catheters are inserted into the trachea through the mouth or nose in order to maintain an open air passage. In medical practice, endotracheal tubes are used to support respiration and to establish and maintain airflow of oxygen and carbon dioxide to ensure adequate gas exchange. Endotracheal tubes require a specific method of insertion through the mouth (orotracheal) or nose (nasotracheal). Endotracheal tubes are usually made from soft plastic material and have certain flexibility to navigate through the tracheal opening. Conversely, a tracheostomy tube is generally a curved metal or rigid plastic tube to be inserted into a tracheostomy stoma (hole) to maintain an open lumen.

Double-walled tracheostomy tubes have been used for ventilating patients for more than 20 years. See, for example, U.S. Pat. No. 5,218,957. In these tubes, a tubular outer portion and a tubular inner portion exist in the form of a lining. The inner portion is placed in such a way that it permits die withdrawal of the inner tubular portion when a build-up of secretion has occurred. The inner portion further helps laminar gas flow through the tube. This method helps keep the airway open in case of a biofilm buildup.

The existing flexible protective sleeves with antimicrobial properties only minimize the accumulation of bacteria on the external surface of the endotracheal tube while the tube is withdrawn in the protective sleeve. While the foregoing and other existing sleeve designs may overcome some of the problems involved in secretion buildup in tracheostomy tubes (and as a means to manipulate the gas flow direction), there are no anti-fouling sleeves for endotracheal tubes adapted to facilitate sensor placement and yet reduce biofilm formation and secretions.

What is needed is an anti-fouling sleeve for an endotracheal tube that protects the trachea during ventilation, not only reducing the possibility of bacterial infections, but also helping to maintain an adequate gas flow as well as to reduce biofilm accumulation.

### BRIEF DESCRIPTION OF THE DRAWINGS

FIG. 1 is a side perspective view of the anti-fouling sleeve according to the present invention shown as inserted into an endotracheal tube inside the trachea of a patient

FIG. 2A is a detail view of the anti-fouling sleeve according to the present invention showing a preferred material of construction for sleeve during insertion of the anti-fouling sleeve into an endotracheal tube.

FIG. 2B is a detail view of the anti-fouling sleeve according to the present invention showing a preferred material of construction for sleeve after insertion of the anti-fouling sleeve into an endotracheal tube.

FIG. 3A is a perspective view of the anti-fouling sleeve according to one embodiment the present invention from the bottom representing the placement of optional sensors 14 and apertures 16 thereon.

FIG. 3B is a side cross-sectional view of the anti-fouling sleeve according to one embodiment of the present invention shown after insertion into an endotracheal tube and depicting detail of the optional tear-away perforations 19.

FIG. 4 depicts side cut-away and cross sectional views of the placement tool 20 for the anti-fouling sleeve according to one embodiment of the present invention.

### SUMMARY OF THE INVENTION

These and other objects are accomplished herein by an anti-fouling sleeve for indwelling catheters such as, for example, an endotracheal tube, a method of placement, and the tools for placement. The sleeve may be disposable and serves as an inner lining or sheath that can be removed from the catheter during use without removing the main lumen, such that an open airway through the patient's trachea is maintained at all times. The sleeve acts as a barrier between the outer lumen and any biological or other material that may accumulate thereon, and can be replaced from time to time to clean and/or dispose of it so that the level of accumulation can be controlled.

The anti-fouling sleeve occupies the entire length of endotracheal tube, and can be installed permanently or made removable and disposable. The sleeve may be instrumented with sensors and/or a UV light source to reduce and potentially eliminate biofilm formation. Once placed inside the endotracheal tube the sleeve expands to conform to the inner diameter of the tube. After use, any accumulated biofilm on the inner portion of the sleeve is removed leaving the inner portion of the endotracheal tube essentially sterile. Alternatively, the sleeve itself may serve as a media for culturing of the biofilm in the sleeve or subsequent laboratory or microbiological analysis or antimicrobial targeting.

In addition, the invention includes the following features:
a. Features/markings to assist in the alignment of the lining with the outer lumen of the EXT. The markings are visible using direct visualization, are radiopaque, or are tactile or other structural features of the lining that may be cooperatively aligned with similar features on the outer lumen for proper alignment. In one embodiment, the structural feature is a ventilation hole.
b. One or more sensors for use by a technician. The sensor(s) may be a flow or pressure sensor, or use spectroscopy or colorimetry techniques to determine the buildup of secretion, biofilm or bacteria on the surface of the lining. Alternatively, the sensor may send out an alert based on duration of use. The sensor(s) may be used to alert the technician as to when the lining needs to be removed, assist the technician in adjusting ventilator settings to achieve proper airflow, etc.
c. A Murphy “Hole” or “eye.”
d. A structure that enhances the structural stability/strength of the ETT or outer lumen.
e. A structure that changes form during insertion into or removal from the ETT or outer lumen, or which has breakaway features.
f. A structure composed of biocompatible material
g. A structure having a friction coefficient conducive to placement/removal or maintenance of position within the ETT/outer lumen.

A structure adapted to coupling with other medical equipment such as a mechanical ventilator.

Other objects, features, and advantages of the present invention will become more apparent from the following detailed description of the preferred embodiment and certain modifications thereof.

### DETAILED DESCRIPTION OF THE PREFERRED EMBODIMENTS

The present invention is an anti-fouling sleeve for indwelling catheters such as, for example, an endotracheal tube for intubation, a method of placement of same inside an endotracheal tube, and the tools for placement.

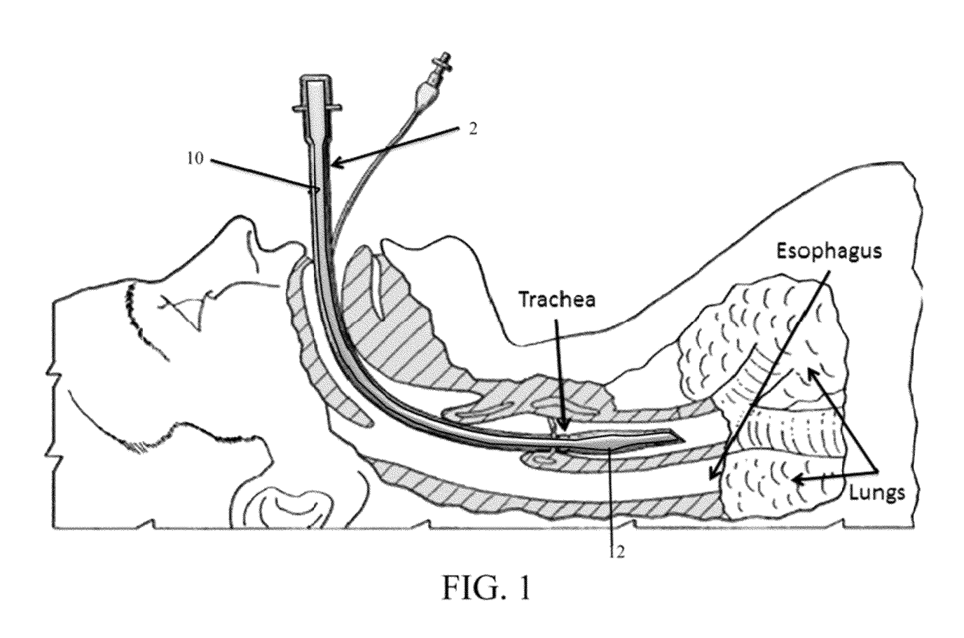

As shown in FIG. 1, the sleeve 10 when deployed inside an exemplary endotracheal tube 2, occupies the entire length of endotracheal tube 2, and can be installed permanently or made removable and disposable, in both cases enabling instrumentation of the endotracheal tube 2 with sensors and/or a UV light source, among other things, while reducing and potentially eliminating biofilm formation. The sleeve 10 is made-up of a flexible, biocompatible, and conformable material to allow it to be placed within the endotracheal tube 2 using a number of potential techniques. The sleeve may have a woven construction, as described below, or alternatively may be formed of a non-woven material, such as extruded or injection-molded plastic having sufficient softness characteristics to enable the features set forth below.

Suitable insertion techniques may include unrolling sleeve 10 inside tube 2, pulling it through, twisting it through or untwisting it inside, or the like. Specifically, the sleeve 10 can be inserted by the means of rolling over the inner wall of the endotracheal tube 2 or using a twisting motion as if screwing into the tube 10, among other approaches. Once placed inside tube 2 the sleeve 10 expands to conform to the inner diameter of the tube 2 thereby covering the entire inner surface of tube 10. The expansion can be accomplished by forming the natural diameter of the sleeve 10 slightly larger than inner diameter of the endotracheal tube 2 such that a preload results from being radially compressed. Alternatively, the sleeve 10 diameter may be controlled by either mechanically manipulating mesh or using an electrically active (shape memory alloy, piezo, magnetostrictive, etc) braided mesh to aid in insertion and withdrawal. This expansion also imparts a radial preload to the endotracheal tube 2 to assure a proper form-fit and encourage adherence to the wall of the tube 2, thus minimizing accumulations between the sleeve 10 and tube 2. The radial preload also helps to reinforce the wall of the tube 2.

In a preferred embodiment, the outer surface of the sleeve 10 is pre-coated with a biocompatible adhesive and/or lubricant. The endotracheal tube 2 may be a conventional tube made from soft plastic material having adequate flexibility to navigate through the tracheal opening. This is contrasted to a tracheostomy tube which is a generally a curved metal or rigid plastic tube inserted into a tracheostomy stoma to maintain an open lumen. The endotracheal tube 2 may be inserted in a conventional manner through the mouth (orotracheal) or nose (nasotracheal). After use, if the disclosed sleeve 10 is disposable, any accumulated biofilm on the inner portion of the sleeve 10 will be removed with the sleeve thereby leaving the inner portion of the endotracheal tube 2 essentially sterile.

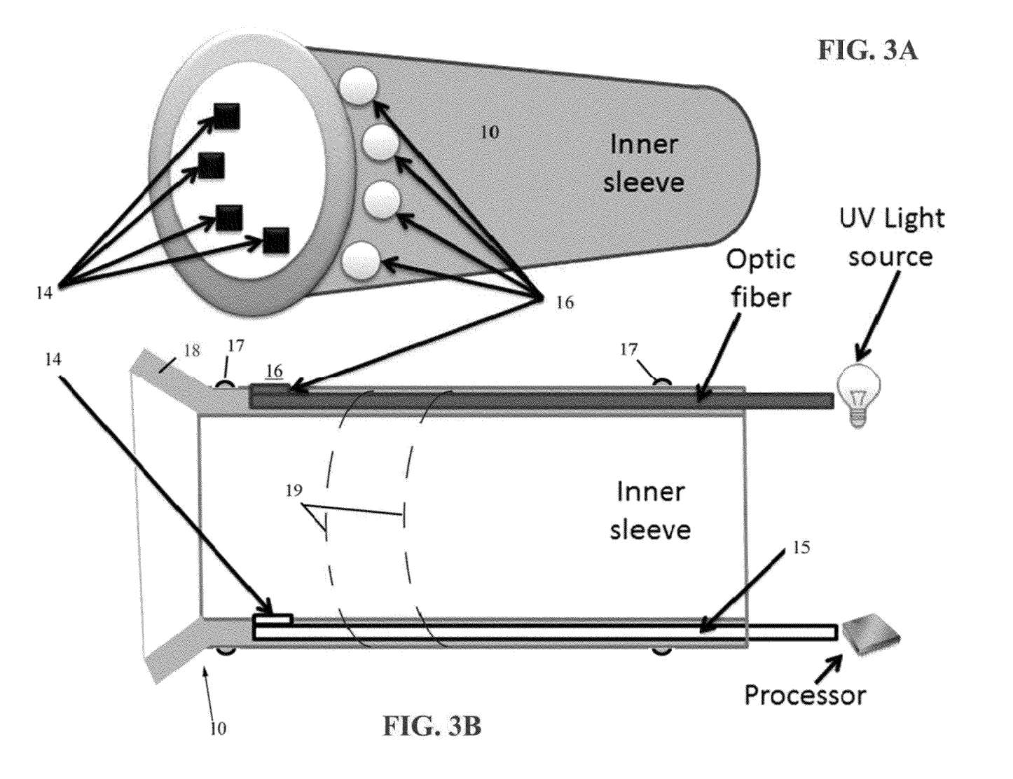

Also for the purposes of cleaning the endotracheal tube, the disclosed inner sleeve 10 is capable of transmitting UV light through one or more optical fibers embedded inside the sleeve 10 and exiting through corresponding fiber optic openings, continuing to an external ultraviolet (UV) light source (as described below). High-intensity ultraviolet light is commonly used for disinfecting, and ultraviolet light fixtures are commonly used in labs and healthcare facilities. Such UV light sources can be coupled to the inner sleeve 10 for transmission through the optical fiber(s) embedded inside the sleeve 10. The optical fiber(s) are terminated within exteriorly-disposed apertures 16 (see FIG. 3) outside the sleeve 10 facing the endotracheal tube 2. This way, UV light is directed onto the interior surface of the endotracheal tube 2 by the fiber optic cable(s). Apertures 16 may be lensed for light dispersion.

The sleeve 10 is generally configured for insertion into the indwelling catheter but has enlarged section 18 at the proximal end that limits insertion. The enlarged section 18 is preferably funneled out at the inward distal end (see also FIG. 3), and this funnel 18 can be extended outside of the endotracheal tube towards the lungs such that it ensures less distal displacement. This funnel 12 also provides a controlled build-up location for secretions which can then be sterilized with UV as above or removed by a suction mechanism.

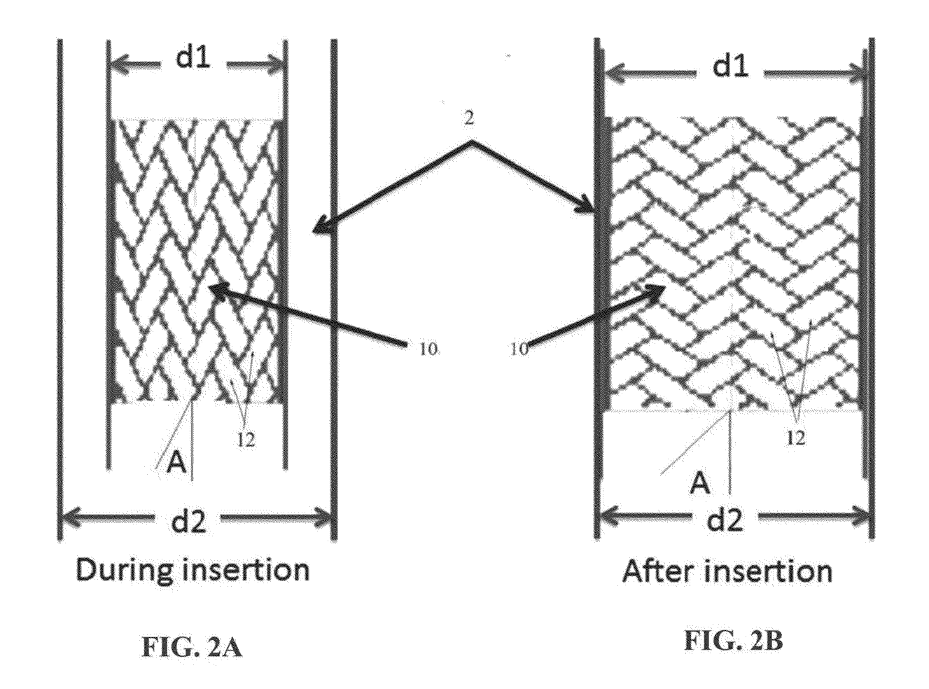

FIG. 2 illustrates a preferred material of construction for sleeve 10 to facilitate expansion. The material may be a braided web preferably comprising fiber filaments 12 braided in a helical fashion to form a sleeve that can expand or contract in diameter. The filaments 12 may be embedded into a soft (e.g. elastomer or rubber) matrix to maintain the spacing between fibers. In a preferred embodiment the filaments 12 are thermally-activated shape-memory fibers such as Nickel-titanium alloys, which, when exposed to the nominal human body temperature, expand. Alternatively, sleeve 10 may be embedded with a non-fiber helical wire (like a simple spring) or mesh that slightly decreases diameter when tensioned to allow the sleeve 10 to be narrowed for insertion and/or removal.

During the insertion process, the outer diameter (d1) of sleeve 10 is less than the inner diameter (d2) of the endotracheal tube 2 to ease the insertion process, as shown at FIG. 2(A). After the insertion process is completed, the braided web of sleeve 10 thermally expands and conforms to the full inner diameter (62) of the endotracheal tube 2, as seen at FIG. 2(B). The sleeve 10 material expands only in radial dimension without increasing in length due to the thermal-expansion property of the material and a proper braid angle A, shown in FIG. 2(A&B). The sleeve 10 filament density (distance between filaments 12) and initial braid angle A of the filaments 12 of sleeve 10 can be varied to influence the stiffness, force generation, deflection range, and other properties of the sleeve 10. The initial braid angle of the filaments 12 is defined as the angle between a braid filament 12 and the radial axis of the sleeve 10.

In addition or alternative to funnel 18 the sleeve 10 may be equipped with insertion indicia to assist in the alignment of the sleeve 10 with the outer lumen of the endotracheal tube 2. The indicia may comprise print markings on the sleeve 10 that are visible using direct visualization, or radiopaque markings visible during imaging. The indicia may be surface features to provide tactile alignment. For example, FIG. 3 depicts raised dimples 17 on the surface of the sleeve 10 that may be cooperatively aligned with conforming holes along the endotracheal tube 2 including, for example, vent holes (known as Murphy holes) as commonly formed in such devices. In addition to or as an alternative to dimples 17, the sleeve 10 may be formed with its own Murphy hole that aligns with that of the endotracheal tube 2 when inserted. The indicia may also inform the technician whether or not there is a sleeve 10 in the tube 2 (otherwise a clear sleeve 10 inside a clear tube 2 could be missed).

The sleeve 10 is preferably formed with a failsafe breakaway seam to allow it to be torn out of the endotracheal tube 2 in case it becomes stuck. This is accomplished with a pre-scored/perforated pattern 19 (see FIG. 3) going helically down the length such that beyond a certain tensile force sleeve 10 will tear away like a ribbon.

The preferred embodiment of the sleeve 10 is instrumented with one or more physical and/or chemical sensors to enable monitoring of a variety of biometric parameters including, but not limited to, temperature, pressure, humidity, pH, oxygen, or flow rate. The ability to detect changes in flow, resistance and pressure drops along the length of sleeve 10 and at the proximal and distal ends helps during weaning trial assessments (i.e., is the tube 2 increasing resistance and causing failure to wean from mechanical ventilation?). Similarly, a back pressure gauge on the proximal end of the sleeve 2 allows assessment of pressure reflected back to the tube 2 after a breath is delivered and is likewise useful in characterizing resistance.

FIG. 3 illustrates a plurality of sensors 14 exposed inwardly around the inner surface of sleeve 10, and each connected by a data transmission wire 15, which may be woven into the braided web. The one or more sensors 14 are connected to a programmable control system to provide operational feedback. Optionally, the connection between the sensor(s) and the control system may be wireless. The sensor(s) 14 may include any one or more from among the following types of sensors:
respiratory function sensor, such as an optical fiber using Bragg grating (FBG) sensing for or monitoring respiratory function;
a movement/position sensor to detect respiration or attempted respiration based on movement of the rib cage or diaphragm, whether that movement be displacement, velocity, or acceleration;
an air flow sensor to detect respiration or attempted respiration based on air flow within the respiratory system, including, but not limited to, the nasal cavity, mouth, trachea, and bronchioles;
a temperature sensor to detect respiration or attempted respiration based on temperature change of a portion of the patient's body;
an oxygen sensor to detect airway oxygen levels.

In a preferred embodiment, the one or more sensors 14 includes a bioburden sensor for sensing the amount of biological growth (“bioburden”) on endotracheal tube 2 inner surface (e.g., at the outside of sleeve 10). The bioburden sensor is similarly connected by cable or fiber configured to the control system in order to alert clinicians as to when changing the sleeve 10 is needed, and/or when changing the sleeve 10 isn't enough and changing of the entire tube 2 is necessary. There are a variety of direct-sensing bioburden sensors available for wound care applications. For example, sleeve 10 may include one or more fiber optic cables (such as cables 16, described below, or an additional set of cables specifically for this purpose) running longitudinally down its length or axially around its circumference. The one or more fiber optic cables (not shown) may be disposed on the interior surface of sleeve 10 or, alternatively, embedded in the sidewall of sleeve 10 and exposed to the interior of sleeve 10 at specified intervals through gaps or windows in the sleeve 10 lining. The points of exposure for the fiber optic cables may additionally be notched to encourage any biofilm that would tend to accumulate on the inside surface of sleeve 10 to accumulate at the areas of exposure of the fiber optic cable. Biofilm accumulation could thus be measured by the degree of impedance of light transmittal along the length of the cable, which could be measured by a connected control system. UV, white, or other light sources could be used within the fiber optic cable to measure accumulation. Alternatively, a simple conducting wire could be substituted for the fiber optic cable, wherein biofilm accumulation is measured in relation to the degree of electrical impedance through the wire. As a less-expensive alternative to direct sensing, the bioburden sensor 14 may be a simple timer, pre-calibrated to time the foregoing intervals in days/hours/minutes, etc. Time could be indicated by a running clock within the attached control system, wherein the technician resets the timer each time he/she places and/or replaces the sleeve 10. Alternatively or in addition to a running timer, an audio, visual or other indicator could alert the technician when a pre-specified interval of time has passed, such as the amount of time that it takes for an unacceptable level of biofilm to accumulate in the sleeve 10 for the average patient, as determined during clinical trials or the like. A visual sensor could be of the type disclosed by U.S. Pat. No. 6,452,873, disclosing a substrate that changes color after a specified time of exposure to air/gas/light/etc., or any other type of photochemical sensor known in the art. Another inexpensive alternative is a sensor that measures weight or load of sleeve 10 or tube 2 to indicate overall accumulation, including biofilm.

As mentioned above, sleeve 10 is preferably equipped with one or more embedded optical fibers 16 that transmit ultraviolet light, as shown in the cross-section of FIG. 3(B). The optical fiber(s) 16 may likewise be woven into the braided web, and each extends to an outwardly exposed fiber optic terminus which may include a lens for outward irradiation of the tube 2.

The optical fibers(s) 16 are connected to an external UV light source, such as a UV LED, which emits UV radiation at an antimicrobial wavelength selected between 170 nm to 300 nm. This light is transmitted through the sleeve 10 via optical fiber(s) 16. The UV light is emitted radially on the outer surface of the sleeve 2 thereby providing 360 degrees of coverage on the inner wall of the endotracheal tube 2 to minimize and avoid secretion buildup due to bacterial growth. The biometric sensors 14 are connected to an external processing unit. A remote power supply or local power source, such as a rechargeable battery, may be provided to power the afore-mentioned components.

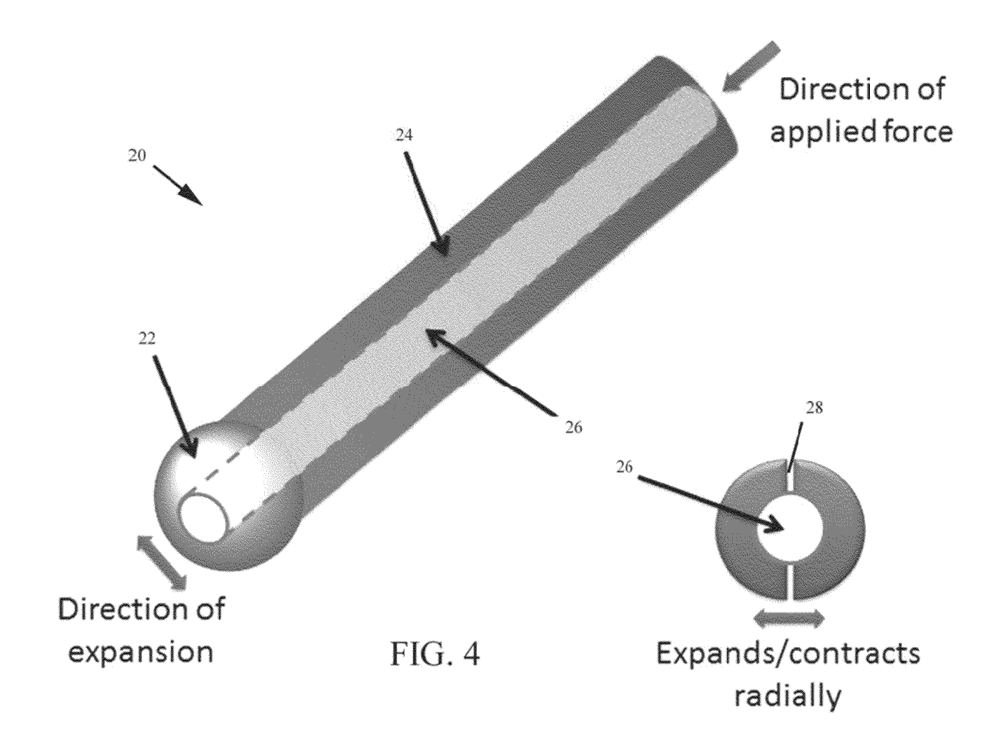

The preferred embodiment of the placement tool 20 for sleeve 10 is shown in FIG. 4. The placement tool comprises a partial-spherical or cylindrical head piece 22 attached to a flexible elongate body 24. Throughout the placement tool 20, there exists an air passageway 26 so that the air flow during the placement process can be maintained. The placement tool 20 must pass through endotracheal tube 2 and therefore the largest diameter of the tool 20 at head piece 22 shall not be significantly larger than that of the tube 2 for which sleeve 10 is placed in. As seen in the cross-section inset of FIG. 4 the head piece 22 and elongate body 24 may be defined by one or more lengthwise notches to facilitate expansion (two being shown). The placement tool 20 may be a made from soft plastic material having adequate flexibility to navigate through the tracheal opening, except the head piece 22 that may be made from hard plastic. The tool 20 may be used for any of the aforementioned insertion techniques, such as unrolling sleeve 10 inside tube 2, pulling it through, twisting it through or untwisting it inside, or the like. During the insertion process, the tool 20 is inserted through tube 2 up to the distal end while sleeve 10 is attached on it. The tool 20 can expand in the radial direction as shown in FIG. 4 by the means of an applied perpendicular force to ensure that sleeve 10 becomes in direct contact with tube 2. It is possible to envision that only the head piece 22 or only the body piece 24 has expansion capability. The expansion can be made possible by a variety of ways such as a scissor mechanism, a pressure induced balloon mechanism, a spiral mechanism, or the like. The tool 20 may contract back before being withdrawn from tube 2, by either removing the applied force or reversing the applied force. In another embodiment, it is also conceivable not to have a head piece 22, and only the body piece 24 is used for insertion process.

To facilitate ease of deployment of sleeve 10, sleeve 10 may be manufactured or fitted to incorporate a groove along its length for cooperative engagement with the deployment tool 20, such that tool 20 can influence the tension, expansion, and lateral or other movement of the sleeve 10 at its distal end or along its entire length. Such a groove may also be useful for cooperative engagement with a cleaning tool (not shown) for cleaning of the sleeve 10 without removal if desired.

It should now be apparent that the above-described sleeve 10 serves as a barrier against biological or other material accumulation, and can be easily be replaced from time to time to clean and/or dispose of it so that the level of accumulation can be controlled. Alternatively, the sleeve 10 itself serves as a convenient media for culturing of any biofilm in the sleeve, or subsequent laboratory or microbiological analysis or antimicrobial targeting thereof.

Having now fully set forth the preferred embodiments and certain modifications of the concept underlying the present invention, various other embodiments as well as certain variations and modifications thereto may obviously occur to those skilled in the art upon becoming familiar with the underlying concept. For example, the same concept and configurations may be implemented in an outer sleeve for an indwelling catheter (rather than inner), providing many of the same benefits and advantages. It is to be understood, therefore, that the invention may be practiced otherwise than as specifically set forth herein.

I claim:

1. An inner sleeve adapted for placement on an inner wall of an indwelling catheter for avoiding secretion and biofilm build-up on surfaces of said catheter, said inner sleeve comprising a flexible tubular liner configured for insertion inside said indwelling catheter and having an open distal end and a proximal end, a length between said proximal and distal ends configured for insertion into said indwelling catheter, and an enlarged section at said proximal end that limits insertion into said indwelling catheter.

2. The inner sleeve according to claim 1, wherein said indwelling catheter has a side vent hole, and said inner sleeve comprises a surface feature for alignment with said side vent hole to ensure full insertion.

3. The inner sleeve according to claim 1, wherein said inner sleeve is formed of a biocompatible material.

4. The inner sleeve according to claim 3, wherein said inner sleeve is formed of a braided mesh.

5. The inner sleeve according to claim 1, wherein said inner sleeve is formed with a score line to allow disassembly in a predetermined fashion.

6. The inner sleeve according to claim 5, wherein said disassembly occurs to facilitate extraction of said inner sleeve from said catheter.

7. The inner sleeve according to claim 6, wherein said disassembly occurs at a predetermined tensile force.

8. The inner sleeve according to claim 5, wherein said disassembly occurs to facilitate laboratory or microbiological analysis or antimicrobial targeting.

9. The inner sleeve according to claim 5, wherein said score line runs helically down said inner sleeve.

10. The inner sleeve according to claim 1, wherein said inner sleeve is formed of an expandable material.

11. The inner sleeve according to claim 1, wherein the enlarged section at said proximal end is funnel-shaped.

12. The inner sleeve according to claim 1, wherein said sleeve imparts a radial preload to said catheter.

13. The inner sleeve according to claim 1, further comprising a biocidal coating on said sleeve.

14. The inner sleeve of claim 1, wherein said sleeve covers the entire interior wall of the indwelling catheter.

15. The inner sleeve of claim 1, wherein said sleeve is disposable.

16. The inner sleeve of claim 1, wherein the sleeve material is flexible, biocompatible and conformable.

17. The inner sleeve of claim 15, wherein said sleeve comprises plastic.

18. The inner sleeve of claim 1, wherein said sleeve material comprises of nickel-titanium alloys.

19. An inner sleeve adapted for placement on an inner wall of an indwelling catheter for avoiding secretion and biofilm build-up on surfaces of said catheter, said inner sleeve comprising a flexible tubular liner configured for insertion inside said indwelling catheter and having an open distal end and a proximal end, a length between said proximal and distal ends configured for insertion into said indwelling catheter, said inner sleeve further comprising a sensor.

20. The inner sleeve of claim 19, further comprising a cable embedded in said flexible tubular liner.

21. The inner sleeve of claim 20, wherein said sensor is connected to said cable and is selected from among a group comprising temperature, pressure, humidity, pH, tissue oxygen, flow rate, O2 and CO2, and light sensors.

22. The inner sleeve of claim 20, wherein said cable is a fiber optic cable to transmit light.

23. The inner sleeve of claim 22, further comprising a light source in optical communication with said at least one fiber optic cable.

24. The inner sleeve of claim 23, wherein the light source is a source of ultraviolet light.

25. The inner sleeve of claim 24, wherein the light source emits UV radiation at an antimicrobial wavelength within a range of 170 nm to 300 nm.

26. The inner sleeve of claim 23, wherein the light source comprises an LED.

27. The inner sleeve of claim 26, wherein the light source comprises an ultraviolet LED.

28. The inner sleeve of claim 22, wherein said sleeve has a fiber optic opening and the light is radiated on said indwelling catheter through said opening.

29. The inner sleeve of claim 19, further comprising a processor to record measurements from said sensors.

30. The inner sleeve of claim 20, further comprising a processor to record measurements from said sensors, said at least one cable extending from said at least one sensor to said processor, wherein measured data are transmitted from said sensors to said processor through said cables.

31. The inner sleeve of claim 19, wherein said sleeve has a braided structure.

32. The inner sleeve of claim 19, wherein the at least one sensor includes a sensor for sensing accumulation on the inner sleeve or indwelling catheter

33. The inner sleeve of claim 32, wherein the sensor optically senses accumulation on the inner sleeve or indwelling catheter.

34. The inner sleeve of claim 32, wherein the sensor comprises a timer for estimating accumulation by passage of time.

35. The inner sleeve of claim 32, wherein the sensor electrically senses accumulation on the inner sleeve or indwelling catheter.

36. The inner sleeve of claim 32, wherein the sensor is non electrical.

37. The inner sleeve of claim 36, wherein the sensor is chemically or photochemically reactive.

38. A method of placing an inner sleeve on an indwelling catheter, comprising the step of: deploying said sleeve on the inner surface of said indwelling catheter.

39. The tool for placing the inner sleeve of claim 1 by the method of claim 38.

40. The tool of claim 39, wherein said tool can expand and contract in radial direction.

41. The tool of claim 40, wherein said expansion and contraction is achieved due to an applied perpendicular force.

42. The method of claim 38, further comprising applying a twisting motion to the inner sleeve.

43. The inner sleeve according to claim 3, wherein said inner sleeve is formed of a woven biocompatible material.

44. The inner sleeve according to claim 1, wherein said inner sleeve further comprises a helical structural element to cause said inner sleeve to contract in diameter when tensioned.

45. The inner sleeve according to claim 1, further comprising an external timer to indicate the length of time since placement of the inner sleeve.

46. The inner sleeve according to claim 1, further comprising a feature or plurality of features to interface with an insertion tool, extraction tool, or suction catheter.

47. The inner sleeve according to claim 1, further comprising a fiber optic cable in optical communication with a light source, wherein the light source emits UV radiation at an antimicrobial wavelength within a range of 170 nm to 300 nm.
